(19) United States
(12) Patent Application Publication
(10) Pub. No.: US 2018/0117855 A1
(43) Pub. Date: May 3, 2018

(54) Integrated Production of Abdominal Aortic Stents Using 3D Printing

(71) Applicants: Michael L. Girou, Plano, TX (US); Frank Henry Markey, Dallas, TX (US); Richard Thomson Bowman, Plano, TX (US); Renee Girou, Plano, TX (US)

(72) Inventors: Michael L. Girou, Plano, TX (US); Frank Henry Markey, Dallas, TX (US); Richard Thomson Bowman, Plano, TX (US); Renee Girou, Plano, TX (US)

(21) Appl. No.: 15/338,344

(22) Filed: Oct. 29, 2016

(51) Int. Cl. B29C 67/00; A61F 2/82; A61F 2/07; B33Y 10/00; B33Y 50/02; B33Y 80/00; G05B 19/4099

(52) U.S. Cl. CPC B29C 67/0088; A61F 2/82; A61F 2/07; B29C 67/007; A61F 2002/061; B33Y 50/02; B33Y 80/00; G05B 19/4099; A61F 2240/002; B33Y 10/00

(57) ABSTRACT

An integrated, automated method to make various abdominal aortic stents with 3D printing technology and computerized recognition algorithms. Cost, risk, and time from assessment to recovery are reduced.

### BACKGROUND

The data from a Computed Tomography (CT) scan is a collection of two-dimensional slices that are stored in a Digital Imaging and Communications in Medicine (DICOM)-format file that can be viewed with open-source software. FIG. 4 shows a three-dimensional (3D) reconstruction of an abdominal CT scan. The mathematics associated with CT is quite sophisticated and advances rapidly. Occasionally, Magnetic Resonance Imaging (MRI) is used instead of CT, but it is better suited for soft organs, but the data files produced follow the DICOM standard in either event.

Three-dimensional (3D) printing is still in its infancy, and the devices range from consumer units using inexpensive filaments and costing hundreds of dollars to industrial systems using special resins and costing almost a million dollars. Our techniques are intended for, but not limited to, devices costing one to three thousand dollars.

3D printer filaments have rapidly evolved in recent years. Today, it is possible to purchase filaments of varying flexibility and strength, some of which have been approved by the Food and Drug Administration for implantation in the human body. Additionally, there are at least three different sources of human-skin replacement filaments.

The abdominal aorta can be thought of as a pipe that carries blood from the heart to the legs and lower body. FIG. 1 shows the location of the abdominal aorta in the body. The abdominal aorta is normally 2 cm in diameter. An abdominal aortic aneurysm (AAA) is defined as a swelling, typically fifty percent or more, of the abdominal aorta. AAA is a concern because it may rupture or hemorrhage. Here are some key notions about an aneurysm:
It can burst, with a mortality rate of 50 to 90%.
It can rupture, in which case the bleeding is internal and slower than with bursting, but still likely to be fatal, and
It is a prime location for embolisms (clots) to form, which can break loose and travel to other places where they block the flow of blood (stroke, heart attack, etc.).
Each year, USA physicians diagnose 200,000 cases of AAA. Not all require intervention. Roughly 15,000 people in the USA die each year from AAA, the 17^{th} leading cause of death in the United States. Physicians often detect this condition during a physical exam when they push on the abdomen and feel a lump that pulsates. Ultrasound will confirm the problem and a Computer-aided Tomography (CT) scan will provide the details.
Approximately one in every 250 people over the age of 50 will die of a ruptured AAA.
AAA affects as many as eight percent of people over the age of 65.
Males are four times more likely to have AAA than females.

There were no treatments until the 1950's, when surgeons started replacing the weakened/swollen part of the artery with a graft. There are several major problems with this open surgery procedure:
The aorta is clamped, both above and below the problem area, so the surgeon has to operate quickly when doing the graft.
The recovery time is several months, several weeks of which are in the hospital (an incision is made from the breastbone down to the pubic bone).
Great expense, typically in the area of several hundred thousand dollars.
High surgical risk, particularly for elderly patients.

One emerging alternative, frequently referred to as a standard abdominal aortic stent, is to insert a stent-graft by applying a catheter to an incision in the groin area, and implant it into the problem area from the inside, similar to inserting a pipe into the interior of the artery. FIG. 2 shows a stented AAA. This takes the pressure on the damaged walls. FIG. 3 shows such a stent. These stents are made by at least two manufacturers, and typically cost fifteen thousand dollars each. Unfortunately, they may fail because the geometry of the problem area is unsuitable: there has to be room above and below the aneurysm for the graft to grow into the skin, and the renal arteries need line up with each other so that the area is covered by the stent without blocking the renal arteries.

The main concerns with the traditional stent approach, as opposed to the old open surgery solution where the patient is cut from the breastbone to the pubic bone and the arterial section is removed and replaced by a graft, are:
The failure to seal at the top and bottom of the graft without occluding the renal arteries.
Traditional stents are solid sheaths, so they block off all the smaller arteries between their top and bottom. This includes four lumbar arteries, two genital/ovarian arteries, and the inferior mesenteric artery. Surgeons are well away of this problem, and rely on collateral blood circulation to the blocked areas to minimize pain and maintain patient functionality. When there is not enough collateral circulation, special bypass grafts are sutured, increasing cost and risk.
Cost of the stent itself, typically fifteen to twenty thousand dollars.
The stents are not customized for a patient.

Fenestrated stent grafts are an emerging technology, and their use has been largely limited to the United Kingdom and a few United States research centers. Here are the features of this approach:
An experienced interventional radiologist manually takes critical measurements from a CT scan of the patient.
The measurements are transmitted to a centralized production facility, typically one that operates an expensive Zenith manufacturing system.
The main body of the stent is produced with holes (fenestrations) through which branch vessels can be inserted.
Then the branch vessels are created, providing a path into the secondary arteries.

There are several drawbacks to fenestrated abdominal aortic stents:
They traditionally require the talents of a skilled interventional radiologist, with attendant delays and opportunity for communications errors.
They are very expensive, typically fifty thousand dollars.
They require the use of expensive specialized equipment, which is located in a limited number of sites.
The overall process is not integrated, thus potentially error-prone, and does not easily allow intervention in the design process by the cardiologist or vascular surgeon.
The time period between medical diagnosis and remediation can be lengthy.

### SUMMARY

We have created an integrated, automated method to make various abdominal aortic stents with 3D printing technology and computerized recognition algorithms. Cost, risk, and time from assessment to recovery are reduced.

### DRAWINGS—FIGURES

FIG. 1 shows the position of the abdominal aorta in the body.

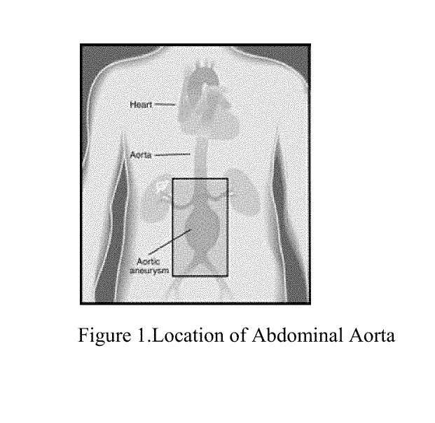

FIG. 2 shows the abdominal aortic aneurysm, with and without stent. An aneurysm is an abnormal swelling of the vessel, typically fifty percent or more.

FIG. 3 shows a free-standing abdominal aortic stent.

FIG. 4 shows a rendering of the abdominal aorta created by merging slices of a CT scan.

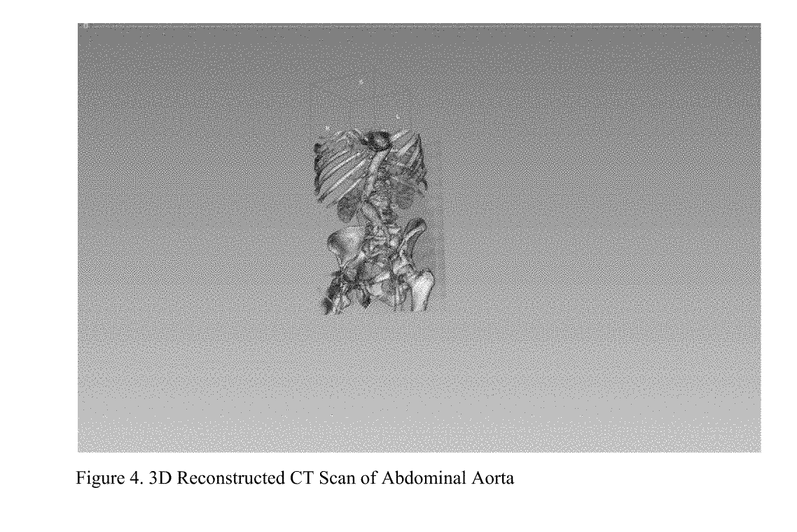

FIG. 5 shows the impact of prime memory bank allocation.

FIG. 6 shows the overall flow of the integrated approach.

### DETAILED DESCRIPTION—FIRST EMBODIMENT

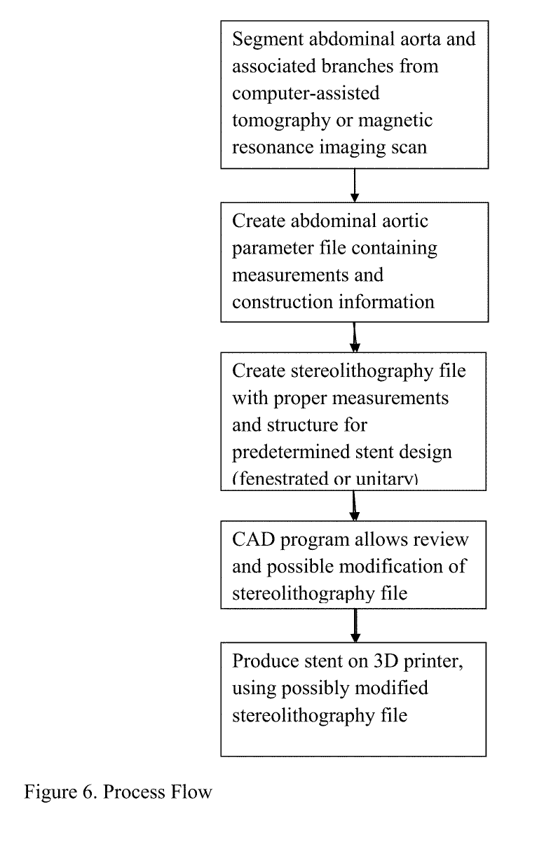

This embodiment applies an integrated approach to programmatically obtaining patient-specific artery locations, and then producing a one-piece, unitary stent, our new alternative to traditional and fenestrated stents, with a 3D printer. FIG. 6 shows the overall flow of the process. The key notion of a unitary stent is to build a sleeve with roots into branch arteries, allowing the unobstructed flow of blood. This is reminiscent of fingers in a glove, and it is a way to introduce stents without blocking key arteries to the brain. The utilization of inexpensive 3D printers, coupled with the economies of scale associated with medically-acceptable filaments, reduces the expected cost of a customized stent to several hundred dollars or less.

Our approach is driven by two sources of information. One source is a front-end program, typically a Graphical User Interface based on a file of standard values, which can be overridden by the physician. The file contains values such as which arteries are candidates for rooting the stent, the depth of the penetration into those arteries, the diameter(s) of the main branch of the stent, and other manufacturing information. The other source is computer code using a Prime Matrix Approach to implement the Segmentation Algorithm.

### The Prime Matrix Approach

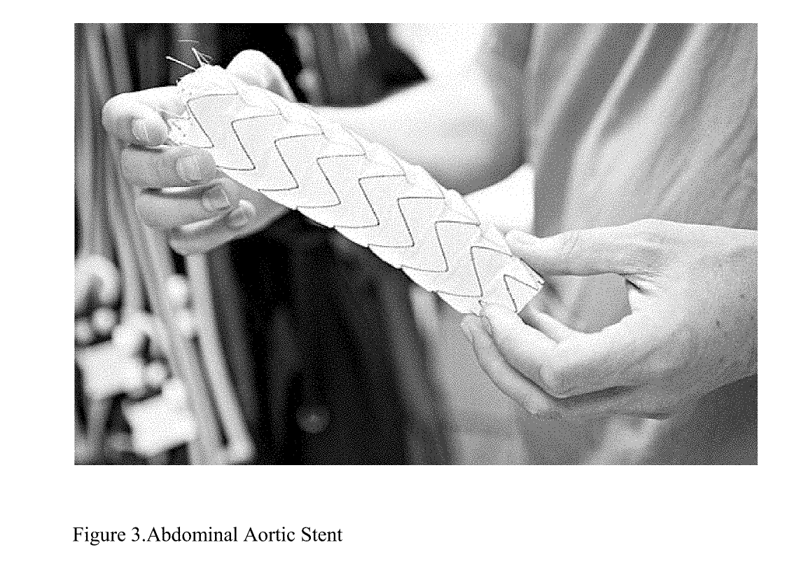

Our techniques, like most medical imaging techniques, are compute-intensive and matrix-based. Modern Computer Processing Units (CPU) and Graphics Processing Units (GPU) have multiple cores that do the computing, but it is not easy to always ensure that these units always have enough data available so that they are always doing useful computing. For example, consider the 4×4 matrix shown in FIG. 3 being processed by a 4-core unit. Depending on the order in which the data is stored, row or column, the cores will be able to simultaneously process rows or columns, but not both because of memory contention issues. The problem is a function of the relative primeness of the matrix to the number of cores.

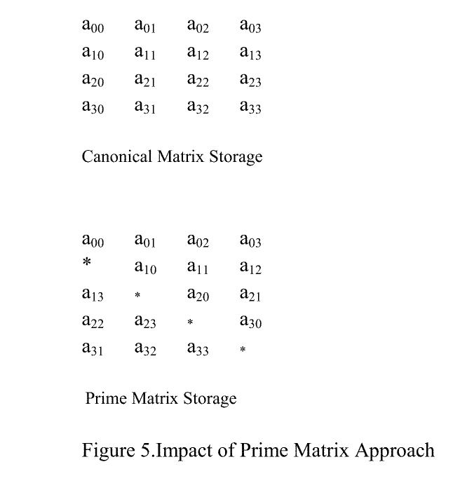

Consider the effect of programmatically representing the 4×4 matrix as a 5×5 matrix, where 5 is the first prime greater than 4. This skewing by embedding dummy elements into the original matrix means that the size of the expanded matrix is always relatively prime to the number of cores, and guarantees that rows and columns can both be simultaneously accessed. This results in 100% utilization of computing power at the expense of a slight increase in total memory required and a bit of delicate programming FIG. 5 shows a typical example.

Note that most CT scanners produce layers of 512×512 matrices, and the first prime number greater than 512 is 521.

This approach is particularly important for our computations, where we are frequently processing both by row and by column, and need to perform those computations on computers normally found in an imaging center or cardiovascular surgery center. We assume that this approach will always be used, without further mention.

### Segmentation Algorithm

Simply stated, the basic algorithm is to construct a model of the blood flow, and then use a method of lines to determine critical points for extracting only the physiological information needed, and then extend the blood flow area to include connected tissue.

First, a parameter file is parsed to obtain processing options, and this is merged with the DICOM header file to get device-specific information.

Then the matrices representing layers are rotated so that they represent frontal views of the patient. Matrix elements with intensity lower than the level indicated in the parameter file are set to zero, speeding up the analysis by eliminating areas that are not sufficiently intense to be part of the abdominal aorta.

Then the blood flow is computed for each layer of matrices, as follows. For each layer, there are two matrices, one reflecting blood intensity (injected dye), and one reflecting the entire abdomen. The result of subtracting the abdominal intensity from the after-die intensity matrix is to isolate the blood portion since the non-blood portion has the same values in both matrices. The resulting layer of matrices will be referred to as the working model.

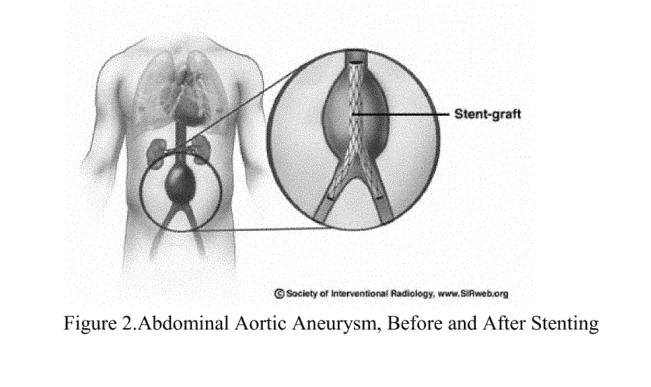

Ideally, the working model containing the abdominal aorta and other blood-containing organs will look like the upside-down Y shape show in FIG. 2. Then, starting at the bottom, we pass a horizontal line through the working model and count the number of intersections. We expect to see four intersections due to the two iliac arteries, tapering off to two intersections where they join together.

Continuing upward from that point, we expect to encounter an increase in the number of intersections as other organs/arteries are encountered, but we will only take the segment contained between the two inner intersections. Unfortunately, that approach needs to be adjusted for several reasons:
There are several minor but important arteries that, when viewed from the front, have a membrane surface that is relatively thick and will show up as points of non-blood flow. In fact, it is important to identify these locations for future surgical considerations.
Sometimes the scanner “hiccups”, or metallic obstacles show up in unexpected locations. In any event, real-life scanner data is not pure.
Either of these issues can obviate the method of lines by introducing false edges.

The solution is to pre-scan each line, paying particular attention to what seems to be a small gap, where ‘small’ is defined in the parameter file. After checking the intensities of other points in the same small area, the program decides when to connect the affected area by changing its non-blood (zero) intensity to that of the average in the neighborhood. Thus, the working model is made to look more like the idealized diagram shown in textbooks.

A side view of the model is processed with the same logic to determine the location of the superior mesenteric artery, the upper limit (modulo the guidance in the parameter file) of the working model.

The working model is then extended by analyzing the non-dye layers, and adding those values that are connected to the working model.

This completes the segmentation phase, and the result is merged with the DICOM header to create a new DICOM file.

### Final Processing

The new DICOM file is then passed to a Computer Aided Design program such as Meshmixer or Blender for review, possible manual revision, and creation of a stereolithography file. This stereolithography file is then passed to a slicer program such as Simplify3D for 3D printing of the unitary stent.

This embodiment solves several problems:
The cost of producing a patient-specific stent is reduced by several orders of magnitude.
It can potentially solve the problem of blocking minor arteries, assuming that the minor artery diameters are large enough to accommodate an inserted stent extension.
A skilled interventional radiologist is not necessarily essential to the embodiment, and the automation of the measurements is accurate, consistent, and the results can transmitted without concern for error to the production portion of the integrated approach.
The required 3D printers are within the budget and amenable to the technical skills of a medical imaging or cardiovascular surgery center.
The algorithm uses a prime memory approach, which allows it to be run on computers found in a medical imaging or cardiovascular surgery center.
The cardiologist or vascular surgeon has the ability to modify the design if desired.
The elapsed time between the initial assessment and the production of a stent is reduced.
The costs, risks, and issues associated with open surgery are obviated.

### DETAILED DESCRIPTION—SECOND EMBODIMENT

The second embodiment is the same as the first embodiment, except that a fenestrated stent is produced rather than a unitary stent. The fenestrated stent uses the same measurements and techniques, augmented by additional configuration file entries for the sizes of the holes and stent branches. This approach is more likely to involve design modification by the cardiologist or vascular surgeon, but it offers another option with the cost-savings and integrated process advantages of the first embodiment.

### DETAILED DESCRIPTION—THIRD EMBODIMENT

The third embodiment is the same as the first embodiment, except that a traditional stent is produced rather than a unitary stent. This traditional stent uses the same measurements and techniques but, but unlike existing traditional stents, it provides a higher degree of patient customization. This approach offers another option with the cost-savings and integrated process advantages of the first embodiment

We claim:

1. A method of using a computer processor and a three-dimensional printer by applying said computer processor to the execution of formulas and logical flow comprising the steps of:
a. the means for performing the segmentation/extraction of a predetermined abdominal aorta and associated branches from a computer-assisted tomography or magnetic resonance imaging scan of said predetermined abdominal aorta and associated branches,
b. the means for creating a parameter file specifying the structure and measurements of said abdominal aorta,
c. the means for creating a stereolithography file, based on said parameter file of said abdominal aorta, for a predetermined stent design for said abdominal aorta,
d. the means for transmitting said stereolithography file to a Computer Aided Design program for manual review and possible modifications, and
e. the means for printing said stereolithography file, possibly modified by said Computer Aided Design program, on said three-dimensional printer,
whereby said steps constitute an integrated, automated approach to producing a plurality of abdominal aortic stents using equipment and skills found in vascular surgical centers.
